(19) United States
(12) Patent Application Publication
(10) Pub. No.: US 2025/0334706 A1
(43) Pub. Date: Oct. 30, 2025

(54) DETECTOR SUPPORTING APPARATUS, MEDICAL IMAGING SYSTEM, AND MEDICAL IMAGING METHOD

(71) Applicant: GE Precision Healthcare LLC, Waukesha, WI (US)

(72) Inventors: Yingying Wang, Beijing (CN); Bin Liu, Beijing (CN); Bingkun Xue, Beijing (CN); Fusheng Li, Beijing (CN); Yuqing Li, Beijing (CN); Mingtao Hu, Beijing (CN)

(21) Appl. No.: 19/189,930

(22) Filed: Apr. 25, 2025

(30) Foreign Application Priority Data
Apr. 29, 2024 (CN) 202410528578.8

(51) Int. Cl. G01T 7/00; A61B 6/00; A61B 6/04

(52) U.S. Cl. CPC G01T 7/00; A61B 6/0407; A61B 6/4452; A61B 6/547

(57) ABSTRACT

Embodiments of the present application provide a detector supporting apparatus, a medical imaging system, and a medical imaging method. The detector supporting apparatus includes: a support frame, for carrying a detector; and a sliding and supporting part, for vertically supporting the support frame at an edge of a bed plate assembly of the medical imaging system and being capable of sliding in the direction of the edge of the bed plate assembly.

### CROSS-REFERENCE TO RELATED APPLICATIONS

The present application claims priority to Chinese Application No. 202410528578.8, filed on Apr. 29, 2024, the entire contents of which is hereby incorporated by reference.

### TECHNICAL FIELD

Embodiments of the present application relate to the technical field of medical imaging, and in particular, to a detector supporting apparatus, a medical imaging system, and a medical imaging method.

### BACKGROUND

In a medical imaging system, emitted X-rays from an X-ray source are directed at a subject under examination and are received by a detector after penetrating the subject under examination. The detector is divided into an array of discrete elements (such as pixels). The detector elements are read to produce an output signal on the basis of the amount or intensity of radiation impinging on each pixel region. The signal is processed to produce a medical image of the subject under examination, and the medical image can be displayed in a display apparatus of the medical imaging system.

X-ray imaging systems currently include an exposure mode in which the position of the detector is freely adjustable, and in which the detector is supported by a support and moved independently of the bed plate or the chest radiography stand, so that the use of grids can be omitted. The application of said mode is particularly necessary in the scenario of lateral image capture of a patient with impaired mobility and in the scenario of image capture requiring the use of a multiple image splicing technique. However, the movement and positioning of the conventional detector support depend on the operator's labor and experience, operation is quite inconvenient, and there is a possibility that the image quality may be affected due to inaccurate positioning of the support.

### SUMMARY

Embodiments of the present application provide a detector supporting apparatus, a medical imaging system, and a medical imaging method.

According to an aspect of the embodiments of the present application, a detector supporting apparatus is provided. The apparatus comprises a support frame, for carrying a detector; and a sliding and supporting part, for vertically supporting the support frame at an edge of a bed plate assembly of a medical imaging system and being capable of sliding in the direction of the edge of the bed plate assembly.

According to an aspect of the embodiments of the present application, a medical imaging system is provided, comprising a bed plate assembly a detector, and the detector supporting apparatus according to the previous aspect.

According to one aspect of the embodiments of the present application, a medical imaging method is provided. The method comprises: carrying a detector by using the detector supporting apparatus according to the previous aspect to slide to a plurality of different positions in the direction of an edge of a bed plate assembly, obtaining medical sub-images of a subject under examination at the plurality of positions, respectively, and splicing the plurality of medical sub-images to obtain a medical image.

With reference to the following description and drawings, specific implementations of the embodiments of the present application are disclosed in detail, and the way in which the principles of the embodiments of the present application can be employed are illustrated. It should be understood that the embodiments of the present application are not limited in scope thereby. Within the scope of the spirit and clauses of the appended claims, the embodiments of the present application include many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and those of ordinary skill in the art may obtain other implementations according to the drawings without involving inventive effort. In the drawings:

FIG. 1 is a schematic diagram of a medical imaging system according to embodiments of the present application;

FIG. 2 and FIG. 3 are schematic diagrams of a detector supporting apparatus according to embodiments of the present application;

FIG. 4 is a schematic diagram of a detector supporting apparatus mounted on a side edge of a bed plate assembly according to embodiments of the present application;

FIG. 5 is a schematic exploded view of a support frame and a sliding and supporting part according to embodiments of the present application;

FIG. 6 is a schematic diagram of markers according to embodiments of the present application;

FIG. 7 is a schematic diagram of a medical imaging method according to embodiments of the present application;

FIG. 8 is a schematic diagram of a support according to embodiments of the present application;

FIG. 9 is a schematic diagram of a transmission part according to embodiments of the present application;

FIG. 10 is a schematic diagram after a support and a transmission part are assembled according to embodiments of the present application;

FIG. 11 is a schematic diagram of a detector supporting apparatus mounted on a side edge of a bed plate assembly according to embodiments of the present application; and

FIG. 12 is a schematic diagram of a medical imaging system according to embodiments of the present application.

### DETAILED DESCRIPTION

The foregoing and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc. are used to distinguish between different elements in terms of appellation, but do not represent a spatial arrangement, a temporal order, or the like of these elements, and these elements should not be limited by these terms. The term “and/or” includes any one of and all combinations of one or more associated listed terms. The terms “include,” “comprise,” “have”, etc., refer to the presence of described features, elements, components, or assemblies, but do not exclude the presence or addition of one or more other features, elements, components, or assemblies. The terms “connect”, “link”, “couple”, etc., used in the embodiments of the present application are not limited to physical or mechanical connections, but may include electrical connections, whether direct or indirect.

In the embodiments of the present application, the singular forms “a” and “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “based on” should be construed as “based at least in part on . . . ”, unless otherwise specified in the context.

The features described and/or illustrated for one embodiment may be used in one or more other embodiments in an identical or similar manner, combined with features in other embodiments, or replace features in other embodiments. The term “include/comprise” when used herein refers to the presence of features, integrated components, steps, or assemblies, but does not exclude the presence or addition of one or more other features, integrated components, steps, or assemblies.

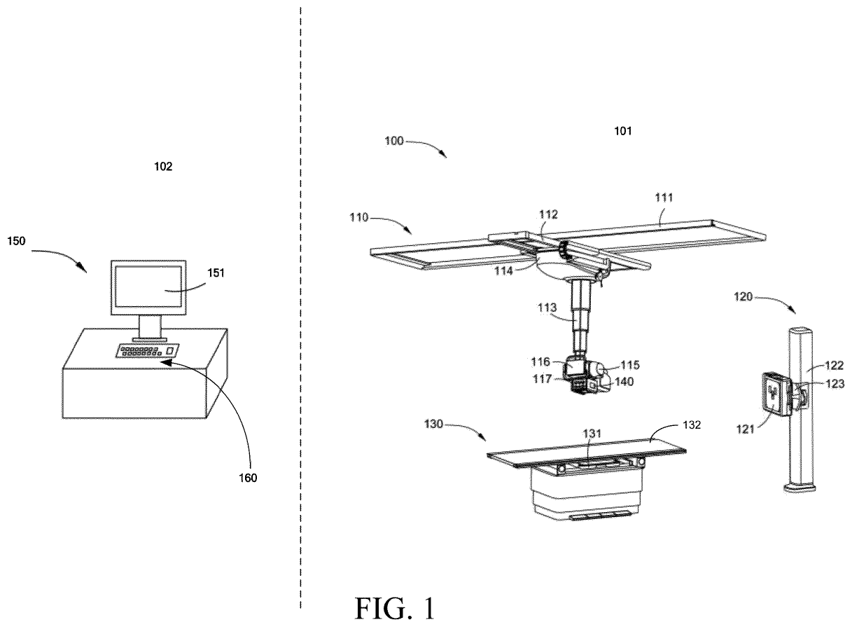

FIG. 1 is a medical imaging system 100 of an embodiment of the present application. As shown in FIG. 1, the medical imaging system 100 includes a suspension apparatus 110, a wall stand apparatus 120 and an examination bed apparatus 130 provided in a scanning room 101, and a control apparatus 150 provided in a control room 102. The suspension apparatus 110 includes a longitudinal guide rail 111, a transverse guide rail 112, a telescopic cylinder 113, a sliding member 114 and a tube assembly 115.

Although some embodiments of the present application are described on the basis of a suspended X-ray imaging system, the embodiments of the present application are not limited thereto.

For ease of description, in the present application, an x-axis, y-axis, and z-axis are defined as the x-axis and the y-axis being located in a horizontal plane and being perpendicular to one another, and the z-axis being perpendicular to the horizontal plane. Specifically, the direction in which the longitudinal guide rail 111 is located is defined as the x-axis, the direction in which the transverse guide rail 112 is located is defined as the y-axis direction, and the direction of extension of the telescopic cylinder 113 is defined as the z-axis direction, and the z-axis direction is the vertical direction.

The longitudinal guide rail 111 and the transverse guide rail 112 are perpendicularly arranged, wherein the longitudinal guide rail 111 is mounted on a ceiling, and the transverse guide rail 112 is mounted on the longitudinal guide rail 111. The telescopic cylinder 113 is used to carry the tube assembly 115.

The sliding member 114 is disposed between the transverse guide rail 112 and the telescopic cylinder 113. The sliding member 114 may include components such as a rotary shaft, a motor, and a reel. The motor can drive the reel to rotate around the rotary shaft, which in turn drives the telescopic cylinder 113 to move along the z axis and/or slide relative to the transverse guide rail. The sliding member 114 can slide relative to the transverse guide rail 112, that is, the sliding member 114 can drive the telescopic cylinder 113 and/or the tube assembly 115 to move in the y-axis direction. Furthermore, the transverse guide rail 112 can slide relative to the longitudinal guide rail 111, which in turn drives the telescopic cylinder 113 and/or the tube assembly 115 to move in the x-axis direction.

The telescopic cylinder 113 includes a plurality of columns having different inner diameters, and the plurality of columns may be sleeved sequentially from bottom to top in columns located thereon to thereby achieve telescoping. The telescopic cylinder 113 can be telescopic (or movable) in the vertical direction, that is, the telescopic cylinder 113 can drive the tube assembly to move in the z-axis direction. The lower end of the telescopic cylinder 113 is further provided with a rotating part, and the rotating part may drive the tube assembly 115 to rotate.

The tube assembly 115 includes an X-ray tube, and the X-ray tube may produce X-rays and project the X-rays to a patient's intended region of interest (ROI). Specifically, the X-ray tube may be positioned adjacent to a beam limiter, and the beam limiter is used to align the X-rays with the patient's intended region of interest. At least part of the X-rays may be attenuated by means of the patient and may be incident on a detector 121/131. In addition, not shown in the drawings, the X-ray imaging system may also include a positionally flexible hand-held detector for imaging some joints or infants.

The suspension apparatus 110 further includes a beam limiter 117, which is usually mounted below the X-ray tube, and the X-rays emitted by the X-ray tube irradiate on the body of a subject under examination by means of an opening of the beam limiter 117. The size of the opening of the beam limiter 117 determines an irradiation range of the X-rays, namely, the size of a region of an exposure field of view (FOV). The position of the X-ray tube and beam limiter 117 in the transverse direction determines the position of the exposure FOV on the body of the subject under examination. It is well known that X-rays are harmful to the human body, so it is necessary to control the X-rays so that the X-rays only irradiate the site of the subject under examination that needs to be examined, namely, the region of interest (ROI).

The suspension apparatus 110 further includes a tube control apparatus (console) 116. The tube control apparatus 116 is mounted on the tube assembly. The tube control apparatus 116 includes user interfaces such as a display screen and a control button for performing preparation work before image capture, such as patient selection, protocol selection, positioning, etc.

The movement of the suspension apparatus 110 includes the movement of the tube assembly along the x-axis, y-axis, and z-axis, as well as the rotation of the tube assembly in a horizontal plane (the axis of rotation is parallel to or coincides with the z-axis) and in a vertical plane (the axis of rotation is parallel to the y-axis). In the described movement, a motor is usually used to drive a rotary shaft which in turn drives a corresponding component to rotate, so as to achieve a corresponding movement or rotation, and a corresponding control component is generally mounted in the sliding member 114. An X-ray imaging unit further includes a motion control unit (not shown in the figure), and the motion control unit can control the described movement of the suspension apparatus 110. Furthermore, the motion control unit can receive a control signal to control a corresponding component to move correspondingly.

The wall stand apparatus 120 includes a first detector assembly 121, a wall stand (e.g., a chest radiography stand) 122, and a connecting portion 123. The connecting portion 123 includes a supporting arm that is vertically connected in the height direction of the wall stand 122 and a rotating bracket that is mounted on the supporting arm, and the first detector assembly 121 is mounted on the rotating bracket. The wall stand apparatus 120 further includes a detector driving apparatus that is provided between the rotating bracket and the first detector assembly 121. Under the drive of the detector driving apparatus, the first detector assembly 121 moves along a direction that is parallel to the height direction of the wall stand 122 in a plane that is supported by the rotating bracket, and the first detector assembly 121 may be further rotated relative to the supporting arm to form an angle with the wall stand. The first detector assembly 121 has a plate-like structure the orientation of which can be changed, so that the incident surface of the X-rays becomes vertical or horizontal depending on the incident direction of the X-rays.

A bed plate assembly 132 and a second detector assembly 131 are included on the examination bed apparatus 130. The second detector assembly 131 includes a moving assembly and an accommodation portion. The moving assembly is used to drive the accommodation portion (e.g., a tray) below the bed plate assembly 132 and a detector panel to move longitudinally along a bed plate. The accommodation portion may be used to accommodate and carry the detector panel. Specifically, the receiving range of the detector panel can be along the longitudinal direction from one side of a bed panel to the other side. Specifically, the moving assembly includes a synchronous belt, a guide rail, and a motor. The guide rail is arranged in the longitudinal direction. The accommodation portion (e.g., the tray) can move relative to the guide rail. One end of the synchronous belt is fixed on the accommodation portion (e.g., the tray), and the other end is connected to the motor, so as to control the synchronous belt by means of the motor to drive the accommodation portion (e.g., the tray) to move, so that the accommodation portion (e.g., the tray) and the detector move in the longitudinal direction. The described movement configuration enables the detector panel to cover the position of the entire bed panel, so that any position on the subject under examination can be imaged, or image splicing can be performed on a plurality of positions.

Alternatively, however, the detector panel in the second detector assembly 131 may be detached from the second detector assembly 131, placed on a separate support or held by the subject under examination, for use in an exposure mode in which the position of the detector is freely adjustable. The selection or use of the first detector assembly 121 and the second detector assembly 131 may be determined on the basis of an image capture site of a patient and/or an image capture protocol, or may be determined on the basis of the position of the subject under examination that is obtained by the capturing of a camera. FIG. 1 merely shows a schematic diagram of a wall stand and an examination bed. It should be understood by those skilled in the art that a wall stand and/or examination bed in any form or arrangement may be selected or just the wall stand can be mounted, and the wall stand and/or examination bed do/does not limit the entire solution of the present application.

In some embodiments, the control apparatus 150 may include a source controller and a detector controller. The source controller is used to command the X-ray source to emit X-rays for image exposure. The detector controller is used to select a suitable detector among a plurality of detectors, and to coordinate the control of various detector functions, such as automatically selecting a corresponding detector according to the position or pose of the subject under examination. Alternatively, the detector controller may perform various signal processing and filtering functions, specifically, for initial adjustment of a dynamic range, interleaving of digital image data, and the like. In some embodiments, the control apparatus may provide power and timing signals for controlling the operation of the X-ray source and the detector.

In some embodiments, the control apparatus may also be configured to use a digitized signal to reconstruct one or more required images and/or determine useful diagnostic information corresponding to a patient, wherein the control apparatus may include one or more dedicated processors, graphics processing units (GPUs), digital signal processors, microcomputers, microcontrollers, application-specific integrated circuits (ASICs), field-programmable gate arrays (FPGAs), or other suitable processing apparatuses.

Certainly, the medical imaging system may also include other numbers, configurations or forms of control apparatuses, for example, the control apparatus may be local (e.g., co-located with one or more medical imaging systems 100, such as within the same facility and/or the same local network). In other implementations, the control apparatus may be remote, and thus only accessible by means of a remote connection (for example, by means of the Internet or other available remote access technologies). In a specific implementation, the control apparatus may also be configured in a cloud-like means, and may be accessed and/or used in a means that is substantially similar to the means by which other cloud-based systems are accessed and used.

The system 100 also includes a storage apparatus (not shown in the figure). A processor may store the digitized signal in a memory. For example, the memory may include a hard disk drive, a floppy disk drive, a CD-read/write drive, a digital versatile disc (DVD) drive, a flash drive, and/or a solid-state memory. The memory may also be integrated together with the processor to effectively use the footprint and/or meet expected imaging requirements.

The system 100 further includes an input apparatus 160. The input apparatus 160 may include a certain form of operator interface, such as a keyboard, a mouse, a voice-activated control apparatus, a touch screen (which may also be used a display apparatus described later), a tracking ball or any other suitable input device. An operator may input an operating signal/control signal to the control apparatus by means of the input device.

The system 100 further includes a display apparatus 151 (such as a touch screen or a display screen). The display apparatus 151 may be used to display an operation interface such as a list of subjects under examination, the positioning or exposure configurations of the subjects under examination, and images of the subjects under examination.

In some embodiments, the medical imaging system may further include an image capture apparatus 140. The subject under examination may be photographed by the image capture apparatus to obtain a captured image that includes the subject under examination, for example a static image or a series of image frames in a dynamic real-time video stream, to carry out auxiliary positioning and exposure configurations and the like. The image capture apparatus may be mounted on the suspension apparatus, for example mounted on a side edge of the beam limiter 117, and the like, and the embodiments of the present application are not limited thereto.

The embodiments of the present application are specifically described below.

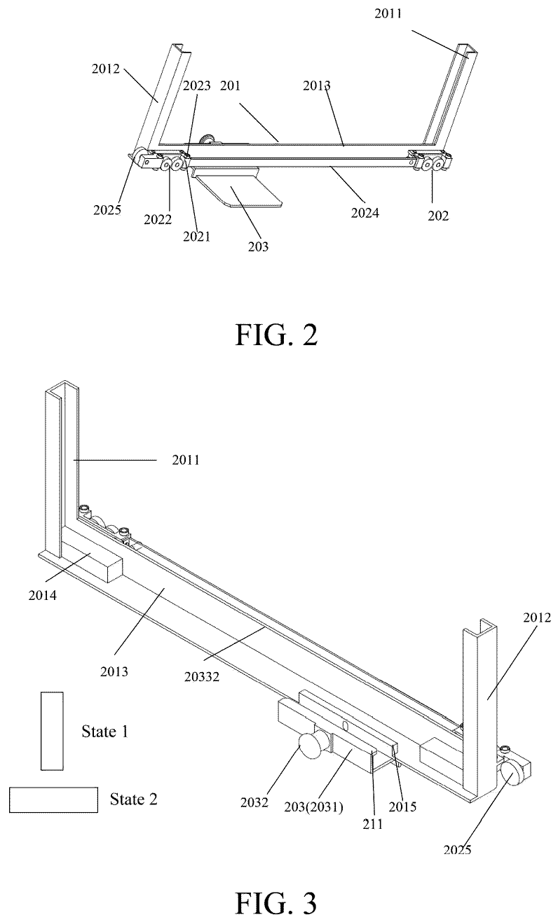

The embodiments of the present application provide a detector supporting apparatus. FIG. 2 and FIG. 3 are schematic diagrams of the detector supporting apparatus according to the embodiments of the present application. As shown in FIG. 2 and FIG. 3, the apparatus includes a support frame 201, for carrying a detector, and a sliding and supporting part 202, for vertically supporting the support frame at an edge of a bed plate assembly of a medical imaging system and being capable of sliding in the direction of the edge of the bed plate assembly.

In some embodiments, the detector carried by the support frame 201 may be detached from the previous second detector assembly 131 or first detector assembly 121. Alternatively, the detector carried by the support frame 201 may be an additionally added detector different from the detector in the second detector assembly 131 or first detector assembly 121, and the embodiments of the present application are not limited thereto.

In some embodiments, the support frame 201 may be a three sided frame. The support frame 201 includes a first frame 2011, a second frame 2012, and a third frame 2013 which are integrally formed. The first frame 2011 and the second frame 2012 extend vertically, for holding side edges of the detector panel. The first frame 2011 and the second frame 2012 are connected by the third frame 2013. The third frame extends horizontally, for holding bottom edges of the detector panel. The length of the third frame 2013 may be the same as the length of the bottom edges of the detector panel. The first frame 2011, the second frame 2012, and the third frame may be U-shaped frames or C-shaped frames, and the embodiments of the present application are not limited thereto.

In some embodiments, the sliding and supporting part 202 is fixed to the edge of the bed plate assembly, but may slide in the direction of the edge of the bed plate assembly, and provides a supporting force, to vertically support the support frame at the edge of the bed plate assembly of the medical imaging system, and to drive the support frame to accordingly slide in the direction of the edge of the bed plate assembly when the sliding and supporting part 202 slides. The direction of the edge of the bed plate assembly may be a side in the length direction, and the embodiments of the present application are not limited thereto.

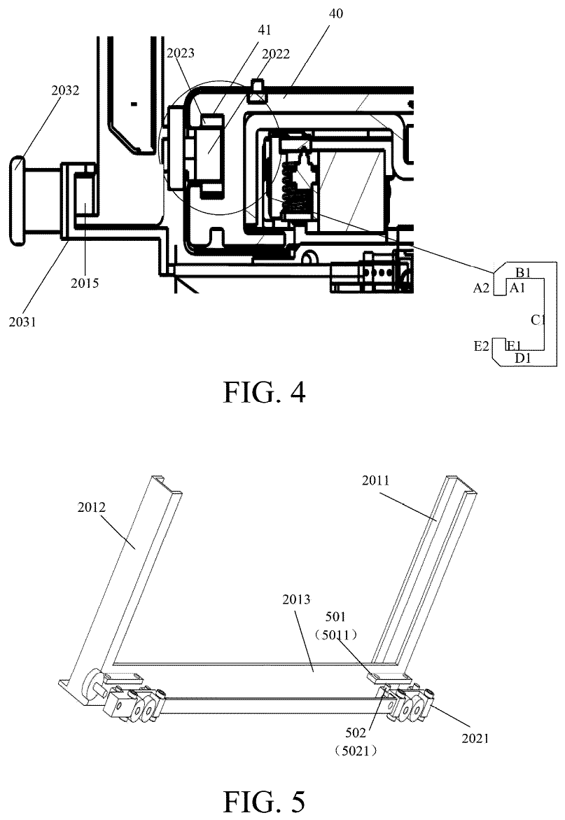

In some embodiments, FIG. 4 is a schematic diagram of the sliding and supporting part 202 mounted on the side edge of the bed plate assembly according to the embodiments of the present application. As shown in FIG. 4, the bed plate assembly 40 includes a bed plate and a frame. The frame may be provided with a groove 41 (the side groove) in the length direction. The groove may be a C-shaped groove, including five inner side walls, i.e., A1, B1, C1, D1, and E1. The sliding and supporting part may be embedded into the side groove and be capable of sliding in the side groove. During mounting, the sliding and supporting part may be pushed into the side groove from one end of the side groove, so that after pushing is completed, the inner side walls A1 and E1 can prevent the sliding and supporting part from falling off from the side groove due to the fact that the groove is C-shaped.

In some embodiments, as shown in FIG. 2 and FIG. 3, the sliding and supporting part 202 includes a sliding portion and a supporting portion. The supporting portion may include a supporting base 2021 and one or more load bearing rollers 2022. The sliding portion may be consisted of one or more guide rollers 2023. The supporting base 2021 may be connected to the third frame 2013. The load bearing roller 2022 is fixed to the supporting base 2021, and the axial direction of the load bearing roller is perpendicular to the direction of the plane where the first frame and the third frame are located. The guide roller 2023 is fixed on the supporting base 2021, and the axial direction of the guide roller is parallel to the extension direction of the first frame. Alternatively, the guide rollers 2023 may be evenly distributed on both sides of the load bearing rollers 2022. After the above-mentioned supporting portion and the sliding portion are pushed into the side groove, at least part of the circumference of the guide roller is in contact with the inner side walls A1 and C1 of the groove 41, and at least part of the circumference of the load bearing roller is in contact with the inner side wall D1 of the groove 41, so as to ensure the sliding and supporting part 202 to smoothly slide in the groove and prevent the sliding and supporting part from falling off from the side groove.

In some embodiments, the sliding portion and the supporting portion may be symmetrically arranged at two ends of the third frame 2013, so as to maintain the balance of the support frame. For example, as shown in FIG. 2 and FIG. 3, each of two ends of the third frame 2013 may be provided with two load bearing rollers and four guide rollers. The four guide rollers are symmetrical with respect to the upper right, the lower right, the upper left, and the lower left of the two load bearing rollers, respectively. The above are merely examples, and the embodiments of the present application are not limited to the number and position of the rollers.

The above embodiments are merely illustrative, and the present application does not limit the structure of the sliding and supporting part 202. For example, a linear guide rail may be further disposed at the edge of the bed plate assembly. The sliding and supporting part 202 may include a slider. The slider is sleeved on the guide rail, to implement that the support frame is vertically supported at the edge of the bed plate assembly of the medical imaging system and is capable of sliding in the direction of the edge of the bed plate assembly. Examples are not listed herein one by one again. In addition, when the groove 41 is not a C-shaped groove, a sliding and supporting function may be implemented by means of making an adaptive change to the structure of the sliding and supporting part. In addition, in FIG. 4, for example, the sliding and supporting part 202 is mounted on the side groove of the bed plate assembly. However, the sliding and supporting part 202 may alternatively be mounted on the position of an edge of a bottom face of the bed plate assembly. For example, the sliding and supporting functions are implemented by means of the above-mentioned structure of the guide rail and the slider. Examples are not listed herein one by one again.

In some embodiments, in order to fix the distance between the sliding portion and the supporting portion on both sides of the third frame, the sliding and supporting part 202 may further include a connecting portion 2024. The connecting portion 2024 presents an elongated shape. The two ends of the connecting portion 2024 are respectively fixed to the supporting bases 2021 on both sides.

In some embodiments, the sliding and supporting part 202 may slide in the direction of the edge of the bed plate assembly under a pushing force of an operator. When the sliding and supporting part 202 is slid to a particular position for image capture, in order to avoid the quality deterioration of a captured image due to the change of the position of the sliding and supporting part 202, the sliding and supporting part may further include a lock portion 2025 for preventing sliding and locking the position. The lock portion 2025 may be a bolt. The supporting base 2021 of at least one of the two ends of the third frame 2013 may be provided with a through hole. An inner side of the through hole is provided with threads matching with the lock portion 2025, so that when the lock portion 2025 is screwed into the through hole and tightened, the edge of the lock portion can be pressed against outer side walls A2 and E2 of the groove 41, thereby achieving the effects of preventing sliding and locking the position. Alternatively, the edges of the lock portion may be provided with a wear-resistant material or texture, so as to further improve the effects of preventing slipping and locking the position and preventing damage to the edges of the bed plate assembly.

In some embodiments, the support frame 201 may be integrally formed with the sliding and supporting part 202, or the support frame 201 may be detachably mounted on the sliding and supporting part 202. FIG. 5 is a schematic exploded view of the support frame 201 and the sliding and supporting part 202 according to the embodiments of the present application. As shown in FIG. 5, the support frame 201 includes a first engaging portion 501, the sliding and supporting part 202 includes a second engaging portion 502, and the support frame is vertically supported at the edge of the bed plate assembly by means of engaging the first engaging portion 501 and the second engaging portion 502. For example, the second engaging portion 502 is fixedly connected to the supporting base 2021, the second engaging portion 502 may be fixed to the supporting base 2021 on the side opposite to the load bearing roller, the second engaging portion 502 may include one or more guide posts 5021, the first engaging portion 501 may be fixed to the third frame 2013, and the first engaging portion 501 may be a flat plate provided with one or more through holes 5011. When the support frame 201 needs to be mounted on the sliding and supporting part 202, the through hole of the first engaging portion 501 is aligned with the guide post of the second engaging portion 502, and the through hole is sleeved around the guide post. When the support frame 201 needs to be detached from the sliding and supporting part 202, the support frame 201 is moved upward, to separate the through hole from the guide post. Therefore, the support frame 201 can be freely detached. Alternatively, the length of the at least one guide post 5021 is greater than or equal to the depth of the through hole 5011, so as to prevent the support frame 201 from falling off from the sliding and supporting part 202.

In some embodiments, as shown in FIG. 3, the support frame 201 (e.g., the third frame 2013) may include a height adjustable spacer 2014. The spacer 2014 is used to carry bottom edges of the detector. For example, spacers 2014 are provided at two ends of the third frame 2013, and the spacers 2014 may be at two heights in a state 1 and a state 2. The minimum height of the spacer at least needs to meet that the bottom edge of the detector is at or slightly above the top surface of the bed plate when the detector is carried, so as to ensure that the entire panel of the detector is located within an imaging region. The height of the detector can be flexibly adjusted by means of the spacer, and the image capture center for different tissue heights/thicknesses can be flexibly adjusted. However, the present application is not limited thereto. For example, a lifting mechanism may further be disposed on the detecting and supporting apparatus, so that the support frame 201 can be lifted or lowered vertically, so as to flexibly adjust the height of the detector. Examples are not listed herein one by one again.

In some embodiments, in existing scenarios such as in which an image splicing technique needs to be used, it is necessary to rely on operator's experience to carry a detector support to a plurality of positions, and therefore the problem of inconsistency in overlapped regions among a plurality of medical sub-images may exist. For the problem, in the embodiments of the present application, one or more markers may be provided on the bed plate assembly, for positioning the detector supporting apparatus, and the plurality of markers may serve as moving positions of the detector for obtaining the medical sub-images, thereby ensuring the consistency of the images and improving the image quality. The one or more markers are capable of sliding in the length direction of the bed plate assembly, so as to accommodate anatomical tissue having different heights/lengths of different subjects under examination.

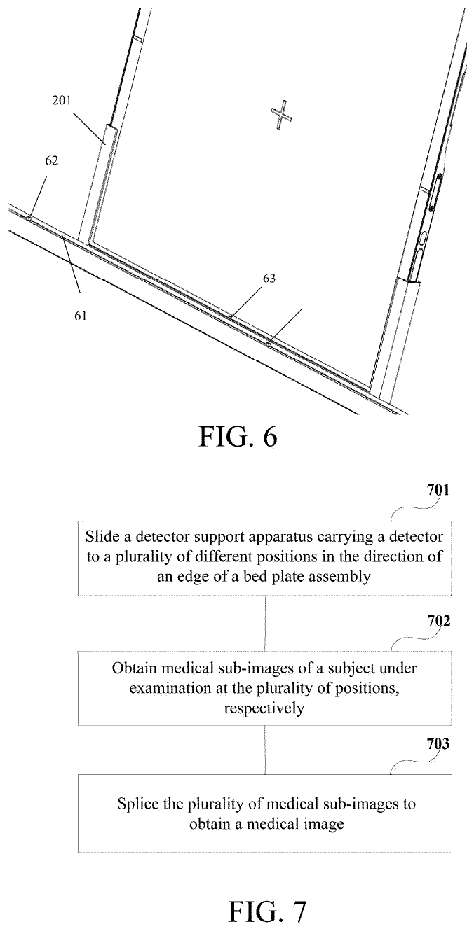

FIG. 6 is a schematic diagram of the markers according to the embodiments of the present application. As shown in FIG. 6, a marker bar 61 may be provided on a side of the bed plate assembly. The marker bar is provided with N markers 62. The number of N may be determined as desired. For example, N=3. When the sliding and supporting part 202 slides under a pushing force of an operator, reference points (e.g., markers 63 provided on the support frame) of the detector supported by the sliding and supporting part may be sequentially aligned with the N markers, so that the detector is sequentially positioned at the N positions, and medical sub-images of the subject under examination are obtained at the N positions, respectively. Alternatively, the marker bar 61 may be disposed in a groove or guide rail on the bed plate assembly, so as to implement sliding in the length direction of the bed plate assembly.

In some embodiments, in order to further optimize the working procedure, save the labor and physical strength of the operator, and improve the positioning accuracy of the detector, the embodiments of the present application further provide a structure that allows the detector supporting apparatus to slide automatically. A detailed description is provided below.

In some embodiments, as shown in FIG. 2 and FIG. 3, the detector supporting apparatus further includes a tracking and connecting part 203, detachably mounted on the support frame, for driving the support frame to automatically moving synchronously with an accommodation portion below the bed plate assembly and capable of carrying the detector. The tracking and connecting part includes: a metallic plate 2031. The metallic plate may be fixed to the support frame and inserted into a gap between the bottom portion of the bed plate assembly and the transmission part described below from the direction of the bottom portion of the third frame. The detector capable of being carried by the accommodation portion is the detector in the previous second detector assembly 131, or may be the same detector as the detector carried by the support frame 201, and the embodiments of the present application are not limited thereto. The accommodation portion can be used to carry the detector, but it does not mean that a detector needs to be actually carried thereon. The accommodation portion may be in a state of not carrying a detector or in a state of carrying a detector, and the embodiments of the present application are not limited thereto.

In some embodiments, the metallic plate 2031 includes a first bent portion 211, the third frame of the support frame is provided with a protruding portion 2015 on the side opposite to the sliding and supporting part, and the first bent portion 211 is hooked on the protruding portion, so as to mount the tracking and connecting part 203 on the support frame. Alternatively, the tracking and connecting part 203 further includes a spring plunger 2032 for mounting the metallic plate 2031 to the support frame. The spring plunger 2032 may be integrally formed with the first bent portion 211 and may be in two states, i.e., an unlocked state and a locked state. During mounting, the sliding and supporting part 202 is first pushed into the groove 41 from one end of the bed plate assembly, the support frame 201 is then mounted on the sliding and supporting part 202, and when the spring plunger is unlocked, the metallic plate is inserted into the gap between the bottom portion of the bed plate assembly and the transmission part described below, and the spring plunger is again locked, so as to fix the metallic plate 2031 on the support frame.

In some embodiments, the apparatus further comprises: a support 204, fixed to the accommodation portion and a transmission part 205 mounted on the support; and when the tracking and connecting part 203 is mounted on the support frame, the transmission part 205 being attracted to the tracking and connecting part. Therefore, when the previous moving assembly drives the accommodation portion below the bed plate assembly to move, the support and the transmission part move synchronously with the accommodation portion, and the attracted tracking and connecting part also moves along with the transmission part, so that the support frame fixed on the tracking and connecting part may automatically move synchronously with the accommodation portion, that is, when the moving assembly provides a driving force, the accommodation portion and the support frame can move synchronously with each other by using the tracking and connecting part.

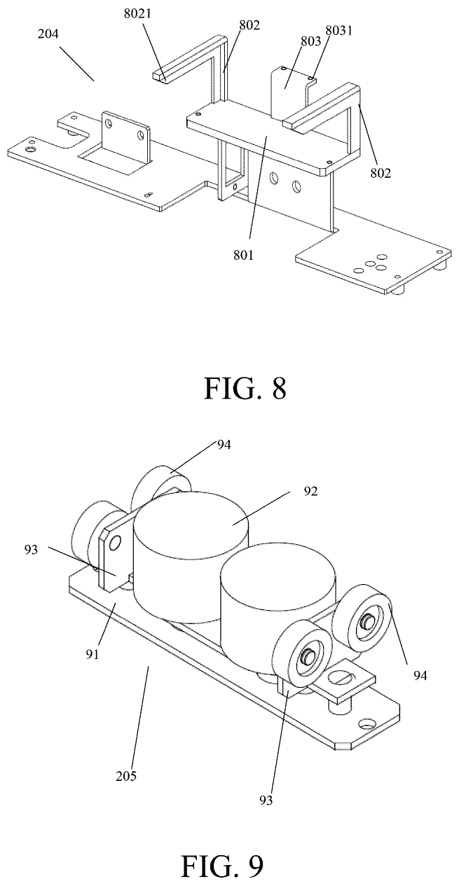

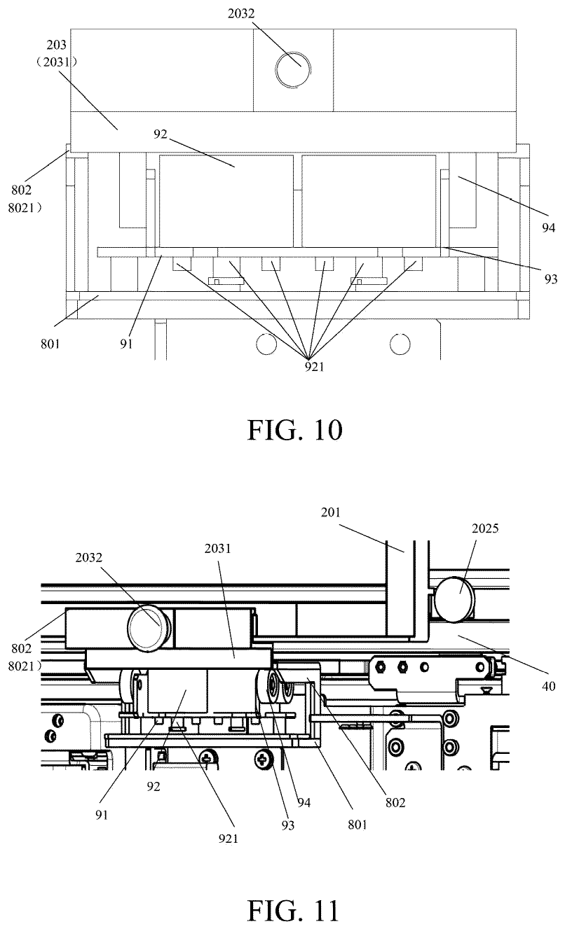

FIG. 8 is a schematic diagram of the support 204 according to the embodiments of the present application. FIG. 9 is a schematic diagram of the transmission part 205 according to the embodiments of the present application. FIG. 10 is a schematic diagram after the support and the transmission part are assembled according to the embodiments of the present application. FIG. 11 is a schematic diagram of the detector supporting apparatus mounted on the side edge of the bed plate assembly according to the embodiments of the present application. As shown in FIG. 8, the support 204 includes a supporting base plate 801 and two supporting arms 802 fixed to the supporting base plate. The support is fixed to the accommodation portion (e.g., directly fixed to a housing of the bed plate) and is capable of synchronously moving with the accommodation portion. When the tracking and connecting part is inserted into the gap between the bottom portion of the bed plate assembly and the transmission part, the supporting arms support the lower surface of the metallic plate of the tracking and connecting part. As shown in FIG. 8, the supporting arms 802 each is provided with a second bent portion 8021, and the step of the second bent portion 8021 can support the lower surface of the metallic plate of the tracking and connecting part. The synchronous movement of the tracking and connecting part can be further ensured by means of the second bent portion 8021. The width of the metallic plate of the tracking and connecting part is approximately the same as the spacing between the two supporting arms 802.

In some embodiments, as shown in FIG. 9 and FIG. 10, the transmission part includes a base 91 and an electromagnetic portion 92 (e.g., made of an electromagnet material). The base 91 is fixed to the supporting base plate 801 (e.g., by means of screw-locking, but not limited thereto). The electromagnetic portion 92 is mounted on the base 91, but not fixed to the base 91. The base 91 is provided with a plurality of holes. A lower portion of the electromagnetic portion 92 is provided with protrusion portions 921 as many as the holes. The protrusion portions 921 are inserted into the holes to mount the electromagnetic portion 92 on the base 91. When an electrical signal is supplied to the electromagnetic portion, the electromagnetic portion generates an electromagnetic force to be attracted to the metallic plate of the tracking and connecting part, and when an electrical signal is not supplied thereto, the electromagnetic portion is separated from the tracking and connecting part due to the gravity of the electromagnetic portion and falls back onto the base 91. The electromagnetic portion 92 may include a spring (not shown) in the middle thereof. When the electromagnetic portion generates an electromagnetic force, the electromagnetic portion is attracted to the tracking and connecting part combined with an elastic force of the spring. When an electrical signal is not supplied, the electromagnetic portion is separated from the tracking and connecting part by the gravity of the electromagnetic portion combined with the elastic force of the spring and falls back onto the base 91. The electromagnetic portion may be a cylindrical electromagnet block, and the embodiments of the present application are not limited to the number and shape of the electromagnetic portion.

How an electrical signal is generated is described below.

In some embodiments, the apparatus may further include a photoelectric sensor (not shown), and the transmission part is electrically connected to the photoelectric sensor and a circuit (board) of the moving assembly. For example, the photoelectric sensor may be mounted on the support frame 803 of the support 204 (the photoelectric sensor is fixed by means of threaded holes 8031 on the support frame 803), and the height of the photoelectric sensor is slightly lower than the plane of the step of the second bent portion 8021, which are merely examples herein, and the embodiments of the present application are not limited thereto. As shown in FIG. 11, when the tracking and connecting part is inserted into the gap between the bottom portion of the bed plate assembly and the transmission part, the photoelectric sensor is shielded by the lower surface of the metallic plate of the tracking and connecting part. When the photoelectric sensor is shielded by an object, a photo-resistor in the sensor is not illuminated. Therefore, the resistance value increases. Two ends of the photo-resistor are at high voltages, the internal circuit of the photo-resistor is equivalently connected, and the electromagnetic portion 92 is equivalently supplied with an electrical signal and generates an electromagnetic force according to the electrical signal to be attracted to the tracking and connecting part. When the photoelectric sensor is not shielded by the lower surface of the metallic plate of the tracking and connecting part (when the metallic plate is not inserted), the light intensity of the photo-resistor in the photoelectric sensor increases. Therefore, the resistance value decreases. Two ends of the photo-resistor are at low voltages, the internal circuit of the photo-resistor is equivalently disconnected, an electrical signal may not be supplied to the electromagnetic portion 92, and therefore an electromagnetic force is not generated.

In some embodiments, alternatively, as shown in FIG. 9 and FIG. 10, the transmission part further includes: support plates 93 located on both sides of the electromagnetic portion and guide portions 94 mounted on the support plates 93. The guide portions 94 may be guide rollers, for guiding the mounting position of the tracking and connecting part. As shown in FIG. 10, the top end of the guide portion 94 is slightly higher than the upper surface of the electromagnetic portion 92, and when the tracking and connecting part is inserted into the gap between the bottom portion of the bed plate assembly and the transmission part, part of the lower surface of the metallic plate of the tracking and connecting part is further in contact with the guide portion 94 and is inserted into the gap between the bottom portion of the bed plate assembly and the transmission part in the direction of rotation of the guide portion 94. The tracking and connecting part can be more easily guided to a correct mounting position by means of the guide portion 94.

The above structures of the tracking and connecting part, the transmission part, and the support are merely examples, the embodiments of the present application are not limited thereto, and any structure that can achieve the above functions falls within the protection scope of the present application.

In the above embodiments, the detector supporting apparatus is capable of sliding in the direction of the edge of the bed plate assembly. The sliding may be manually triggered, or automatically triggered. When the tracking and connecting part is not installed, the sliding is manual, and when the tracking and connecting part is installed, the sliding is automatic. The operator can flexibly switch between a manual sliding mode and an automatic sliding mode by means of mounting or detaching the tracking and connecting part as desired.

Further provided in the embodiments of the present application is a medical imaging method. FIG. 7 is a schematic diagram of a medical imaging method according to the embodiments of the present application. As shown in FIG. 7, the method includes:
701: sliding a detector supporting apparatus carrying a detector to a plurality of different positions in the direction of an edge of a bed plate assembly;
702: obtaining medical sub-images of a subject under examination at the plurality of positions, respectively; and
703: splicing the plurality of medical sub-images to obtain a medical image.

In a manual mode: During mounting, first the sliding and supporting part 202 is pushed into the groove 41 from one end of the bed plate assembly, then the support frame 201 is mounted on the sliding and supporting part 202, the marker bar 61 is moved to a predetermined position according to the specification of the scanning protocols, and the markers 63 on the assembled detector supporting apparatus are sequentially aligned with the N markers 62 on the marker bar 61, so as to slide the detector supporting apparatus to N positions. When moving into alignment with a first marker, the position of the detector supporting apparatus is locked by means of the lock portion, and the positions of the X-ray source and the detector are moved in coordination, so as to obtain a medical sub-image. The position of the detector supporting apparatus is then unlocked by means of the lock portion, the detector supporting apparatus is moved to a next marker, the positions of the X-ray source and the detector are moved in coordination, to obtain a next medical sub-image, and so on, until N medical sub-images are obtained. The N medical sub-images are spliced to generate a medical image. Reference may be made to the related art for the method of splicing, which will not be described herein again.

In an automatic mode: On the basis of the manual mode, the metallic plate 2031 is inserted, to lock the spring plunger 2032. Due to the fact that the detector supporting apparatus and the accommodation portion under the bed plate assembly can move synchronously, according to the set scanning protocols, the moving assembly may drive the accommodation portion to automatically move to a plurality of different positions (a plurality of positions in the length direction of the bed plate), the detector carried by the detector supporting apparatus accordingly moves synchronously to the plurality of positions following the accommodation portion, and the positions of the X-ray source and the detector are moved in coordination, so as to obtain N medical sub-images. The N medical sub-images are spliced to generate a medical image. Reference may be made to the related art for the method of splicing, which will not be described herein again.

The above is only an example, and the embodiments of the present application are not only applicable to a scenario of image splicing, but also applicable to a scenario in which image splicing is not needed. Examples are not listed herein one by one again.

It should be noted that the foregoing accompanying drawings merely schematically illustrate embodiments of the present application, but the present application is not limited thereto. For example, the order of execution of operations may be appropriately adjusted. In addition, some other operations may be added or some operations may be omitted. Alternatively, operations may be added. Those skilled in the art can make appropriate variations according to the above content, rather than being limited by the disclosure of the foregoing accompanying drawings.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

According to the embodiments of the present application, the support frame is vertically supported at the edge of the bed plate assembly of the medical imaging system by means of the sliding and supporting part, and is capable of sliding along the direction of the edge of the bed plate assembly, so that the effort and physical strength of a doctor are saved, the structure of the existing imaging system does not need to be changed, thereby saving costs, and more clinical use (e.g., image splicing) scenarios and special positioning examination can be met.

In addition, due to the fact that the moving assembly is used to drive the accommodation portion (e.g. the tray) below the bed plate assembly and the detector panel to move along the longitudinal direction of the bed plate, and the X-ray source in the medical imaging system is arranged to be automatically aligned (or linked) with the detector below the bed plate assembly, that is, the center of the X-ray source can always be aligned with the detector below the bed plate assembly no matter to which position the moving assembly drives the accommodation portion and the detector capable of being accommodated therein, the detector supporting apparatus according to the embodiments of the present application can move synchronously with the accommodation portion below the bed plate assembly by means of the tracking and connecting part, so that the detector carried by the detector supporting apparatus can be automatically aligned with the X-ray source without calibration. The effort and physical strength of the doctor can be further saved, the positioning of the detector can be performed more precisely, the imaging quality can be improved, and the position of the detector can be automatically changed by using the existing moving assembly without an additional control system.

In addition, the manual sliding mode and the automatic sliding mode can be flexibly switched by means of detaching and mounting of the tracking and connecting part.

In addition, by means of providing one or more markers on the bed plate assembly, the positioning of the detector can be performed more precisely, so that the problem of inconsistency in overlapped regions among a plurality of medical sub-images can be avoided, and the image quality can be improved. The one or more markers are capable of sliding in the length direction of the bed plate assembly, so as to accommodate anatomical tissue having different lengths/heights of different subjects under examination.

In addition, the lock portion is provided on the sliding and connecting part, so that sliding can be prevented and the position of the detector can be locked.

In addition, the support frame includes a first engaging portion, the sliding and supporting part includes a second engaging portion, and the support frame can be freely removed by means of engaging the first engaging portion and the second engaging portion.

In addition, the height of the detector can be flexibly adjusted by means of the spacer on the support frame. In addition, the image capture center of different tissue heights/thicknesses can be flexibly adjusted by means of the spacer on the support frame.

In addition, the tracking and connecting part can be more conveniently guided to the correct mounting position by means of the guide portions on the transmission part.

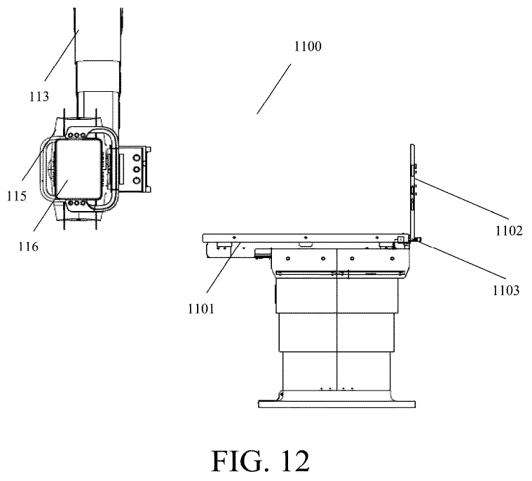

Embodiments of the present application further provide a medical imaging system. FIG. 12 is a schematic diagram of the medical imaging system according to the embodiments of the present application. As shown in FIG. 12, the system 1100 includes: a bed plate assembly 1101; a detector 1102; and a detector supporting apparatus 1103. Regarding the bed plate assembly 1101, reference may be made to the previous embodiments for the implementation of the detector 1102. For example, the detector 1102 may be detached from the second detector assembly 131 or the first detector assembly 121. Alternatively, the detector 1102 may be an additionally added detector different from the detector in the second detector assembly 131 or the first detector assembly 121. In this case, the medical imaging system may simultaneously apply the detector in the second detector assembly below the bed plate assembly and the detector 1102, i.e., using the two detectors simultaneously, to simultaneously capture images of a patient's anteroposterior and lateral positions. In this case, the X-ray source may perform image capture by changing its position back and forth, or the medical imaging system may be provided with two sets of X-ray sources, to respectively coordinate with the two detectors to obtain medical images.

In addition, the bed plate assembly 1101 may further be provided with one or more markers. For the implementation of the detector supporting apparatus 1103, reference may be made to the foregoing embodiments, which will not be repeated here. The medical imaging system may further include other assemblies, for example, a telescopic cylinder 113, a tube assembly 115, and a tube control apparatus 116, etc. For details, reference may be made to FIG. 1. Examples are not listed herein one by one again.

The medical imaging system includes, but is not limited to: a computed tomography (CT) system, a magnetic resonance imaging (MRI) system, a C-arm imaging system, a positron emission computed tomography (PET) system, a single photon emission computed tomography (SPECT) system, an ultrasonic system, an X-ray imaging system, or any other suitable medical imaging system.

Although some embodiments of the present application are described on the basis of the suspended X-ray imaging system shown in FIG. 1, the embodiments of the present application are not limited thereto. For example, the medical imaging system may also be a floor-standing X-ray imaging system, a mobile X-ray imaging system, or the like. Examples are not listed herein one by one again.

The embodiments of the present application further provide a computer-readable program, which, when executed in an apparatus or a medical imaging system, causes a computer to execute, in the medical imaging system, the medical imaging method according to the foregoing embodiments.

The embodiments of the present application further provide a computer program product, at least including a computer-readable program, wherein the computer-readable program causes a computer to execute, in a medical imaging system, the medical imaging method according to the foregoing embodiments.

The above apparatus and method of the present application can be implemented by hardware, or can be implemented by hardware in combination with software. The present application relates to such a computer-readable program that when executed by a logic component, the program causes the logic component to implement the foregoing apparatus or a constituent component, or causes the logic component to implement various methods or steps as described above. The present application further relates to a storage medium for storing the above program, such as a hard disk, a magnetic disk, an optical disk, a DVD, a flash memory, etc.

The method/apparatus described with reference to the embodiments of the present application may be directly embodied as hardware, a software module executed by a processor, or a combination of the two. For example, one or more of the functional block diagrams and/or one or more combinations of the functional block diagrams as shown in the drawings may correspond to either software modules or hardware modules of a computer program flow. The foregoing software modules may respectively correspond to the steps shown in the figures. The foregoing hardware modules may be implemented, for example, by firming the foregoing software modules by using a field-programmable gate array (FPGA).

The software modules may be located in a RAM memory, a flash memory, a ROM memory, an EPROM memory, an EEPROM memory, a register, a hard disk, a removable disk, a CD-ROM, or any storage medium in other forms known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory apparatus, then the software modules may be stored in the MEGA-SIM card or the large-capacity flash memory apparatus.

One or more of the functional blocks and/or one or more combinations of the functional blocks shown in the drawings may be implemented as a general-purpose processor, a digital signal processor (DSP), an application-specific integrated circuit (ASIC), a field-programmable gate array (FPGA) or other programmable logic devices, a discrete gate or transistor logic device, a discrete hardware assembly, or any appropriate combination thereof, which is used for implementing the functions described in the present application. The one or more functional blocks and/or the one or more combinations of the functional blocks shown in the drawings may also be implemented as a combination of computing equipment, such as a combination of a DSP and a microprocessor, a plurality of microprocessors, one or more microprocessors in communication combination with a DSP, or any other such configuration.

The present application is described above with reference to specific embodiments. However, it should be clear to those skilled in the art that the foregoing description is merely illustrative and is not intended to limit the scope of protection of the present application. Various variations and modifications may be made by those skilled in the art according to the principle of the present application, and said variations and modifications also fall within the scope of the present application.

1. A detector supporting apparatus, characterized by comprising:
a support frame, for carrying a detector; and
a sliding and supporting part, for vertically supporting the support frame at an edge of a bed plate assembly of a medical imaging system and being capable of sliding in the direction of the edge of the bed plate assembly.

2. The apparatus according to claim 1, further comprising:
a tracking and connecting part, detachably mounted on the support frame and used for driving the support frame to automatically move synchronously with an accommodation portion which is below the bed plate assembly and is capable of carrying the detector.

3. The apparatus according to claim 2, further comprising:
a support fixed to the accommodation portion and a transmission part mounted on the support;
and when the tracking and connecting part is mounted on the support frame, the transmission part being attracted to the tracking and connecting part.

4. The apparatus according to claim 3, further comprising: a photoelectric sensor;
when the tracking and connecting part is mounted on the support frame, the photoelectric sensor being shielded by the tracking and connecting part and the transmission part being supplied with an electrical signal;
and the transmission part generating an electromagnetic force according to the electrical signal to be attracted to the tracking and connecting part.

5. The apparatus according to claim 2, wherein the tracking and connecting part further comprises: a metallic plate.

6. The apparatus according to claim 5, wherein the tracking and connecting part further comprises a spring plunger for mounting the metallic plate on the support frame.

7. The apparatus according to claim 3, wherein the support comprises a supporting base plate and two supporting arms fixed to the supporting base plate, and when the tracking and connecting part is mounted on the support frame, the supporting arms support the tracking and connecting part.

8. The apparatus according to claim 7, wherein the transmission part comprises an electromagnetic portion; and when an electrical signal is supplied thereto, the electromagnetic portion generates an electromagnetic force to be attracted to the tracking and connecting part, and when an electrical signal is not supplied thereto, the electromagnetic portion is separated from the tracking and connecting part.

9. The apparatus according to claim 8, wherein the transmission part further comprises: guide portions, located on both sides of the electromagnetic portion and used for guiding a mounting position of the tracking and connecting part.

10. The apparatus according to claim 1, wherein the support frame comprises a first engaging portion, the sliding and supporting part comprises a second engaging portion, and the support frame is vertically supported at the edge of the bed plate assembly by means of engaging the first engaging portion with the second engaging portion.

11. The apparatus according to claim 1, wherein the support frame comprises a height adjustable spacer for carrying bottom edges of the detector.

12. The apparatus according to claim 1, wherein the sliding and supporting part is embedded in a side groove of the bed plate assembly and is capable of sliding in the side groove.

13. The apparatus according to claim 1, wherein the sliding and supporting part further comprises a lock portion for preventing sliding and locking the position.

14. The apparatus according to claim 1, wherein the bed plate assembly is provided with one or more markers, for positioning the detector supporting apparatus.

15. The apparatus according to claim 14, wherein the one or more markers are capable of sliding in the length direction of the bed plate assembly.
